(19) United States
(12) Patent Application Publication
(10) Pub. No.: US 2005/0249743 A1
(43) Pub. Date: Nov. 10, 2005

(54) Isolated peptides which bind to HLA-A24 molecules and uses thereof

(76) Inventors: Thierry Boon-Falleur, Brussels (BE); Pierre Van Der Bruggen, Brussels (BE)

Correspondence Address: FULBRIGHT & JAWORSKI, LLP, 666 FIFTH AVE, NEW YORK, NY, 10103-3198, US

(21) Appl. No.: 11/032,498

(22) Filed: Jan. 10, 2005

Related U.S. Application Data
(60) Provisional application No. 60/535,751, filed on Jan. 12, 2004.

(51) Int. Cl. A61K 39/395; C07H 21/04; C07K 14/47; C07K 7/08; C07K 7/06

(52) U.S. Cl. 424/185.1; 514/12; 514/13; 514/14; 514/15; 530/324; 530/325; 530/326; 530/327; 536/23.5

(57) ABSTRACT

Peptides which consist of amino acid sequences found in MAGE-4 bind to HLA-A24 to form T cell epitopes. The therapeutic and diagnostic ramifications of this are the subject of this invention, as are various products obtained in the course of the development of the invention.

### FIELD OF THE INVENTION

This application claims priority from U.S. Provisional Patent Application Ser. No. 60/535,751 filed Jan. 12, 2004, which is incorporated herein by reference in its entirety.

This invention relates to peptides which form immunologically active complexes with MHC molecules. More particularly, it involves peptides based upon amino acid sequences found in the molecule referred to as “MAGE-4,” which form complexes with the MHC molecule HLA-A24.

### BACKGROUND AND PRIOR ART

The study of the recognition or lack of recognition of cancer cells by a host organism has proceeded in many different directions. Understanding of the field presumes some understanding of both basic immunology and oncology.

Early research on mouse tumors revealed that these displayed molecules which led to rejection of tumor cells when transplanted into syngeneic animals. These molecules are “recognized” by T cells in the recipient animal, and provoke a cytolytic T cell response with lysis of the transplanted cells. This evidence was first obtained with tumors induced in vitro by chemical carcinogens, such as methylcholanthrene. The antigens expressed by the tumors and which elicited the T cell response were found to be different for each tumor. See Prehn, et al., J. Natl. Canc. Inst. 18: 769-778 (1957); Klein, et al., Cancer Res. 20:1561-1572 (1960); Gross, Cancer Res. 3:326-333 (1943), Basombrio, Cancer Res. 30:2458-2462 (1970) for general teachings on inducing tumors with chemical carcinogens and differences in cell surface antigens. This class of antigens has come to be known as “tumor specific transplantation antigens” or “TSTAs”. Following the observation of the presentation of such antigens when induced by chemical carcinogens, similar results were obtained when tumors were induced in vitro via ultraviolet radiation. See Kripke, J. Natl. Canc. Inst. 53:333-1336 (1974).

While T cell mediated immune responses were observed for the types of tumor described supra, spontaneous tumors were thought to be generally non-immunogenic. These were therefore believed not to present antigens which provoked a response to the tumor carrying subject. See Hewitt, et al., Brit. J Cancer 33:241-259 (1976).

The family of turn antigen presenting cell lines are immunogenic variants obtained by mutagenesis of mouse tumor cells or cell lines, as described by Boon, et al., J. Exp. Med. 152:1184-1193 (1980), the disclosure of which is incorporated by reference. To elaborate, tum⁻ antigens are obtained by mutating tumor cells which do not generate an immune response in syngeneic mice and will form tumors (i.e., “tum⁺” cells). When these tum⁺ cells are mutagenized, they are rejected by syngeneic mice, and fail to form tumors (thus “tum⁻”) See Boon, et al., Proc. Natl. Acad. Sci USA 74:272 (1977), the disclosure of which is incorporated by reference. Many tumor types have been shown to exhibit this phenomenon. See, e.g., Frost, et al., Cancer Res. 43:125 (1983).

It appears that tur⁻ variants fail to form progressive tumors because they elicit an immune rejection process. The evidence in favor of this hypothesis includes the ability of “tum⁻” variants of tumors, i.e., those which do not normally form tumors, to do so in mice with immune systems suppressed by sublethal irradiation (Van Pel, et al., Proc. Natl, Acad. Sci. USA 76:5282-5285 (1979)) and the observation that intraperitoneally injected tur⁻ cells of mastocytoma P815 multiply exponentially for 12-15 days, and then are eliminated in only a few days in the midst of an influx of lymphocytes and macrophages (Uyttenhove, et al., J. Exp. Med. 152:1175-1183 (1980)). Further evidence includes the observation that mice acquire an immune memory which permits them to resist subsequent challenge to the same tur⁻ variant, even when immunosuppressive amounts of radiation are administered with the following challenge to the same tum⁻ variant, even when immunosuppressive amounts of radiation are administered with the following challenge of cells (Boon, et al., Proc. Natl, Acad. Sci. USA 74:272-275 (1977); Van Pel, et al., supra; Uyttenhove, et al., supra). Later research found that when spontaneous tumors were subjected to mutagenesis, immunogenic variants were produced which did generate a response. Indeed, these variants were able to elicit an immune protective response against the original tumor. See Van Pel, et al., J. Exp. Med. 157:1992-2001 (1983). Thus, it has been shown that it is possible to elicit presentation of a so-called “tumor rejection antigen” in a tumor which is a target for a syngeneic rejection response. Similar results have been obtained when foreign genes have been transfected into spontaneous tumors. See Fearon, et al., Cancer Res. 48:2975-1980 (1988) in this regard.

A class of antigens has been recognized which are presented on the surface of tumor cells and are recognized by cytotoxic T cells, leading to lysis. This class of antigens will be referred to as “tumor rejection antigens” or “TRAs” hereafter. TRAs may or may not elicit antibody responses. The extent to which these antigens have been studied, has been via cytolytic T cell characterization studies, in vitro i.e., the study of the identification of the antigen by a particular cytolytic T cell (“CTL” hereafter) subset. The subset proliferates upon recognition of the presented tumor rejection antigen, and the cells presenting the antigen are lysed. Characterization studies have identified CTL clones which specifically lyse cells expressing the antigens. Examples of this work may be found in Levy et al., Adv. Cancer Res. 24:1-59 (1977); Boon, et al., J. Exp. Med. 152:1184-1193 (1980); Brunner, et al., J. Immunol. 124:1627-1634 (1980); Maryanski, et al., Eur. J. Immunol. 124:1627-1634 (1980); Maryanski, et al., Eur. J. Immunol. 12:406-412 (1982); Palladino, et al., Canc. Res. 47:5074-5079 (1987). This type of analysis is required for other types of antigens recognized by CTLs, including minor histocompatibility antigens, the male specific H—Y antigens, and a class of antigens, referred to as “tum⁻” antigens, and discussed herein.

A tumor exemplary of the subject matter described supra is known as P815. See DePlaen, et al, Proc. Natl. Acad. Sci. USA 85:2274-2278 (1988); Sikora, et al., EMBO J 9:1041-1050 (1990), and Sibille, et al., J. Exp. Med. 172:35-45 (1990), the disclosures of which are incorporated by reference. The P815 tumor is a mastocytoma, induced in a DBA/2 mouse with methylcholanthrene and cultured as both an in vitro tumor and a cell line. The P815 line has generated many turn variants following mutagenesis, including variants referred to as P91A (DePlaen, supra), 35B (Szikora, supra) and P198 (Sibille, supra). In contrast to tumor rejection antigens—and this is a key distinction—the tum⁻ antigens are only present after the tumor cells are mutagenized. Tumor rejection antigens are present on cells of a given tumor without mutagenesis. Hence, with reference to the literature, a cell line can be tum⁺, such as the line referred to as “P1”, and can be provoked to produce tur⁻ variants. Since the tum⁻ phenotype differs from that of the parent cell line, one expects a difference in the DNA of tum⁻ cell lines as compared to their tum⁺ parental lines, and this difference can be exploited to locate the gene of interest in tum⁻ cells. As a result, it was found that genes of tum⁻ variants such as P91A, 35B and P198 differ from their normal alleles by point mutations in the coding regions of the gene. See Szikora and Sibille, supra, and Lurquin, et al., Cell 58:293-303 (1989). This has proved not to be the case with the TRAs of this invention. These papers also demonstrated that peptides derived from the tum⁻ antigen are presented by the L_{d} molecule for recognition by CTLs. P91A is presented by L_{d}, P35 by D^{d} and P198 by K^{d}.

The “MAGE” gene family comprises 24 functional genes divided into three clusters, named MAGE-A, B and C. See DePlaen, et al., Immunogenetics 40:360-369 (1994); U.S. Pat. No. 5,612,201 to DePlaen; Lurquin, et al., Genomics 46:397-408 (1997); Lucas, et al., Cancer Res. 58:743-752 (1998); Chomez, et al., Cancer Res. 61:5544-5551 (2001), all of which are incorporated by reference. U.S. Pat. No. 5,342,774, the disclosure of which is incorporated by reference, disclosed three members of the MAGE family of genes. MAGE-1, 2 and 3 are disclosed therein. Also see Traversari, et al., J. Exp. Med 176:1453-1457 (1993); Science 254:1643-147 (1991), the disclosures of which are incorporated by reference. With respect to MAGE-1, in addition to the '774 patent, see e.g. U.S. Pat. No. 5,925,729. Additional members of the MAGE family, such as MAGE-4, have been discovered and are disclosed in, e.g., DePlaen, supra, and U.S. Pat. No. 5,612,201, supra.

The genes are useful as a source for the isolated and purified tumor rejection antigen precursor and the TRA themselves, either of which can be used as an agent for treating the cancer for which the antigen is a “marker”, as well as in various diagnostic and surveillance approaches to oncology, discussed infra. It is known, for example that tum⁻ cells can be used to generate CTLs which lyse cells presenting different tum⁻ cells can be used to generate CTLs which lyse cells presenting different tum⁻ antigens as well as tum⁺ cells. See, e.g., Maryanski, et al., Eur. J. Immunol 12:401 (1982); and Van den Eynde, et al., Modern Trends in Leukemia IX (June 1990), the disclosures of which are incorporated by reference. The tumor rejection antigen precursor may be expressed in cells transfected by the gene, and then used to generate an immune response against a tumor of interest.

In the parallel case of human neoplasms, it has been observed that autologous mixed lymphocyte-tumor cell cultures (“MLTC” hereafter) frequently generate responder lymphocytes which lyse autologous tumor cells and do not lyse natural killer targets, autologous EBV-transformed B cells, or autologous fibroblasts (see Anichini, et al., Immuno. Today 8:385-389 (1987)). This response has been particularly well studied for melanomas, and MLTC have been carried out either with peripheral blood cells or with tumor infiltrating lymphocytes. Examples of the literature in this area including Knuth, et al., Proc. Natl. Acad. Sci. USA 86:2804-2802 (1984); Mukherji, et al., J. Exp. Med. 158:240 (1983); Hérin, et al., Int. J Canc. 39:390-396 (1987); Topalian, et al., J. Clin. Oncol 6:839-853 (1988). Stable cytotoxic T cell clones (“CTLs” hereafter) have been derived from MLTC responder cells, and these clones are specific for the tumor cells. See Mukherji, et al., supra, Hérin, et al., supra, Knuth, et al., supra. The antigens recognized on tumor cells by these autologous CTLs do not appear to represent a cultural artifact, since they are found on fresh tumor cells. See Topalian, et al., supra; Degiovanni, et al., Eur. J. Immul. 20:1865-1868 (1990). These observations, coupled with the techniques used herein to isolate the genes for specific murine tumor rejection antigen precursors, have led to the isolation of nucleic acid sequences coding for tumor rejection antigen precursors of TRAs presented on human tumors. It is now possible to isolate the nucleic acid sequences which code for tumor rejection antigen precursors, including, but not being limited to those most characteristic of a particular tumor, with ramifications that are described infra.

Additional work has focused upon the presentation of TRAs by the class of molecules known as major histocompataibility complexes, or “MHCs”. This work has resulted in several unexpected discoveries regarding the field. Specifically, in U.S. Pat. No. 5,405,940, the disclosure of which is incorporated by reference, nonapeptides including a MAGE-3 derived peptide, are taught which are presented by HLA-A1 molecules. The reference teaches that given the known specificity of particular peptides for particular HLA molecules, one should expect a particular peptide to bind one HLA molecule, but not others. This is important, because different individuals possess different HLA phenotypes. As a result, while identification of a particular peptide as being a partner for a specific HLA molecule has diagnostic and therapeutic ramifications, these are only relevant for individuals with that particular HLA phenotype. There is a need for further work in the area, because cellular abnormalities are not restricted to one particular HLA phenotype, and targeted therapy requires some knowledge of the phenotype of the abnormal cells at issue.

Additional peptides have been identified which consist of amino acid sequences found in MAGE-1, but which bind to different MHC molecules. See, e.g., U.S. Pat. Nos. 5,405,940 and 5,925,729 which describe peptides which bind to HLA-A1 molecules, and also see U.S. Pat. Nos. 5,558,995 and 6,228,971, which teach peptides consisting of amino acid sequences found in MAGE-1, which bind to HLA-Cw*1601 molecules. CTLs obtained from two melanoma patients after mixed lymphocyte-tumor cell cultures have been found to recognize MAGE-1 based peptides presented by HLA-A1, B37 and Cw16 molecules. See, e.g., Tanzarella, et al., Canc. Res. 59:2668-74 (1999); Traversari, et al., Immunogenetics 35:45-52 (1992); Van der Bruggen, et al., Eur. J. Immunol. 24:2134-2140 (1994), all of which are incorporated by reference in their entirety. Also see, e.g., Fujie, et al., Int. J. Cancer 80:169-172 (1999), who used synthetic peptides, based upon motif analysis such as that taught by Rammensee, et al., Immunogenetics 41:178-228 (1995), to develop synthetic peptides which should be good at binding to different HLA molecules, including HLA-A3 (Chaux, et al., J. Immunol 163:2928-2836 (1999); A68 (Chaux, et al., ibid.); B7 (Luiten, et al., Tissue Antigens 55:149-152 (2000)); B35 (Luiten, et al., Tissue Antigens 56:77-81 (2000)); B53 (Chaux, et al., supra); Cw2 (Chaux, et al., supra); Cw3 (Chaux, et al., supra); DR13 (Chaux, et al., J. Exp. Med. 189:767-78 (1999); and DR15 (Chaux, et al., Eur. J. Immunol 31:1910-6 (2001)).

It is important to note that different approaches have been taken to identifying the peptides described herein with different ramifications. For example, Gaugler et al., J. Exp. Med. 179:921-930 (1994) and Tanzarella, et al., supra, secured CTLs from melanoma patients following autologous, mixed lymphocyte tumor cell cultures. With respect to the other references cited herein, “motif analysis”, using information found in, e.g., Rammensee, supra, incorporated by reference, was applied to the complete sequence of MAGE-1 protein to identify potential HLA molecule binders. These were then tested, and active molecules identified thereby.

This approach, i.e., employing motif analysis, has been found to exhibit a major drawback in that several peptide specific CTL generated using the synthetic peptides, do not recognize HLA matched tumor cells which express MAGE-molecules endogenously. There have been two explanations proposed for this. One is that the peptides at issue are not generated efficiently by the cells' antigen processing and presentation machinery. See Speiser, et al., Cancer Immunity 2:14-19 (2002) The second is that the CTLs obtained using high concentrations of the synthetic peptides have low affinity for the target. See Dahl, et al., J. Immunol 157:239-246 (1996).

MAGE-4 is expressed in a variety of cancers. Data indicate that it is expressed in more than 50% of carcinomas of the esophagus, head and neck, lung and bladder and 6% of carcinomas of the breast. See Rosenberg, S., Principles and Practice of the Biologic Therapy of Cancer, Lippincott Williams & Wilkins, Philadelphia (3rd ed. 2000). Identification of additional MAGE-4 antigenic peptides is important because a number of tumors express MAGE-4 without expressing MAGE-A1 and MAGE-A3. These include carcinomas of the lung, head and neck, esophagus, bladder and breast. There are two known alleles of MAGE-4: MAGE-4a and MAGE-4b. The putative proteins differ by a single amino acid. See DePlaen, et al., Genomics 40:305-313 (1997). It has been shown that two-thirds of the MAGE-4 positive samples expressed MAGE-4b. See Kobayashi, et al., Tissue Antigens 62:426-432 (2003).

A new strategy has been developed for identifying only well processed tumor antigens: dendritic cells transduced with gene MAGE-4 are used as stimulator cells for autologous CD8⁺ T cells. See, e.g., Luiten, et al., Tissue Antigens 55:149-152 (2000); Chaux, et al., J. Immunol 163:2928-36 (1999); Luiten, et al., Tissue Antigens 56:77-81 (2000); Schultz, et al., Tissue Antigens 57:103-109 (2001), and Van den Eynde and van der Bruggen: Cancer Immunity 2001: www.cancerimmunity.org/peptidedatabase/tcellepitopes.html, all of which are incorporated by reference, for examples of the application of this technique, with identification of relevant antigenic peptides.

Marsh, et al., The HLA Factsbook, (Academic Press, 2000), incorporated by reference, supplements older information on MHC binding peptides, such as that provided by Ramensee, supra. Relevant here is Marsh's discussion of the MHC molecule HLA-A24. Marsh, et al. note that approximately 6% of Black, 20% of Caucasian and 42% of Oriental populations present HLA-A24 alleles (twenty subtypes have been identified). Marsh, et al. also disclose that the binding motif for HLA-A24 consists of Y or F at position 2 and F, W, I or L at the carboxy terminus. None of the T cell epitopes disclosed by Marsh, et al. fall within the claimed invention.

As will be seen herein, it has now been observed that a MAGE-4 peptide is a T cell epitope for HLA-A24. This, and the ramifications of this observation, constitute the invention, which is elaborated upon in the detailed description which follows.

### EXAMPLE 1

This example describes experiments in which autologous dendritic cells were generated. Blood samples were taken from a hemochromatosis patient, and peripheral blood mononuclear cells (PBMCs) were isolated by standard density gradient centrifugation. The tubes were centrifuged at 2,200 rpm for twenty minutes at room temperature. The interphase containing the PBMCs was harvested and washed at least three times in cold phosphate buffer solution with 2 mM EDTA in order to remove the remaining platelets.

The washed PBMCs were placed in culture flasks at a density of 2×10⁶ cells per cm² in RPMI 1640 supplemented with HEPES (2.38 g/liter), 1.5 mM L-glutamine (AAG), antibiotics, and 1% autologous plasma that was heat-inactivated at 56° C. for thirty minutes (hereafter referred to as complete RPMI medium). The cells were left to adhere for 1 hour at 37° C.

Non-adherent cells were discarded and adherent cells were cultured in the presence of IL-4 (200 U/ml) and GM-CSF (70 ng/ml) in complete RPMI medium. Cultures were fed on days 2 and 4 by removing one third of the volume and adding fresh medium with cytokines. The cultures were frozen on day 5.

### EXAMPLE 2

This example describes the production of HLA-A24/MAGE-4 and control tetramers.

Recombinant HLA-A2402 molecules produced in E. coli were folded in vitro with beta2-microglobulin and peptide NYKRCFPVI (SEQ ID NO: 1) from MAGE-4, or peptide LYVDSLFFL (SEQ. ID NO: 2) from PRAME (control) (See Ikeda, et al., Immunity 6:199-208 (1997)), in accordance with Altman, et al., Science 274:94-96 (1996). The peptide of SEQ ID NO: 1 corresponds to amino acids 143-151 of MAGE-4. This specific peptide was selected because it is homologous to the peptide NYKHCFPEI (SEQ ID NO: 3), a MAGE-1 encoded peptide previously shown to be recognized by HLA-A24 restricted T cells. See Fujie, et al., Int. J. Cancer 80(2):169-172 (1999). The results of this experiment demonstrated successful production of HLA-A2402 and SEQ ID NO: 1 peptide complexes.

The HLA-peptide complexes were then purified by gel filtration, biotinylated and mixed as described in Altman, et al., supra with extraviden-phycoerythrin (PE) for the HLA-A24/MAGE-4 tetramer, or streptavidin-APC for the PRAME control tetramer. Both extravidin-PE and streptavidin-APC are commercially available. The tetramers were then used to label T cells, as described in the next example.

### EXAMPLE 3

In this experiment, the tetramers produced in Example 2 were used to label CD8⁺ T lymphocytes, and the cells were sorted.

A previously obtained sample containing 4.5×10⁸ B and T cells from an HLA-A2402 individual without cancer was thawed overnight, after which 3×10⁸ viable B and T cells remained. After rosetting according to standard procedures, 1.8×10⁸ purified T cells were obtained.

The T cells were washed, and resuspended at 2×10⁷ cells per ml. in PBS with 1% human serum. Then, they were incubated for 15 minutes at 4° C. with the A24/MAGE-4 tetramers (20 nm) or the A24/PRAME control tetramers (5 nM). Anti CD8⁺ antibodies coupled to FITC were then added to label those T cells expressing CD8 molecule. After further incubation for 15 minutes, the cells were washed.

These tetramer-labeled CD8⁺ labeled T cells (2.5×10⁷ cells/8 μl) were incubated at 4° C. with an anti-PE antibody coupled to magnetic beads (20 μl), according to standard methods. The cells were then washed and enriched by magnetic sorting, also in accordance with standard methods. A total of 2.4×10⁵ tetramer-labeled T cells were recovered.

### EXAMPLE 4

This example describes how the autologous dendritic cells from Example 1 were used to stimulate the CD8⁺ tetramer-labeled T cells.

As discussed supra, autologous dendritic cells had been generated and frozen. These cultures were thawed one day before they were to be used, and 21×10⁶ dendritic cells were incubated overnight with IL-4 (200 μ/ml) and GM-CSF (70 ng/ml). Then, the dendritic cells were incubated for 6 hours with 5 mg/ml of peptide NYKRCFPVI (SEQ ID NO: 1) in the presence of 1 μg/ml of ribomunyl and IFN-γ (500 U/ml) in order to induce their maturation, and washed.

The CD8⁺ tetramer-labeled T cells obtained after magnetic sorting (Example 3) were distributed in 50 U-bottomed microwells (5,000 cells/well) and cultured in 200 μl of IMDM supplemented with gentamicin (15 μg/ml), 1.5 mM L-glutamine (AAG), 10% human serum, IL-2 (100 μ/ml) and IL-7 (5 ng/ml). The CD8⁺ T cells were stimulated with irradiated (100 Gray) peptide pulsed autologous dendritic cells on day 0 (2,000 dendritic cells/well) and day 12 (13,000 dendritic cells/well), in the presence of IL-2 (50 μ/ml) and IL-7 (5 ng/ml).

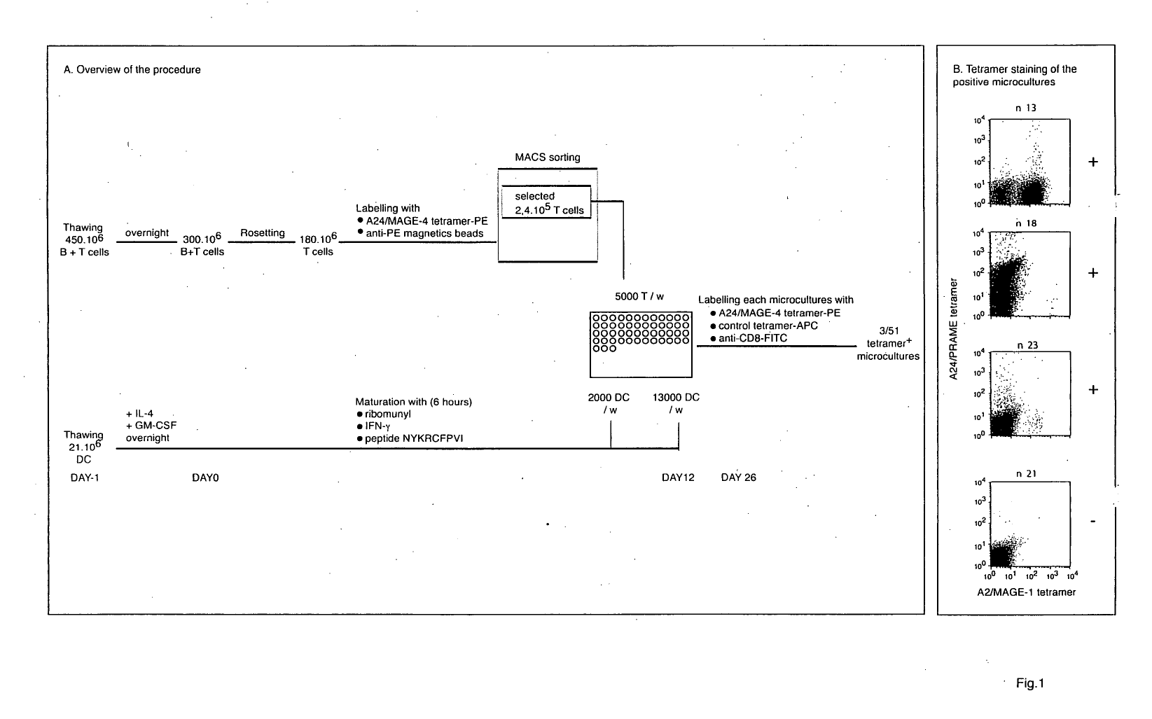

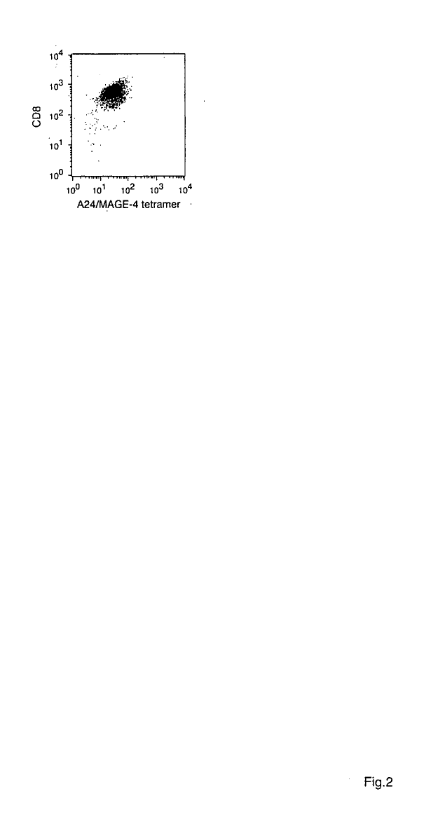

Aliquots of each microculture were tested on day 25 for the presence of T cells that could be specifically labeled with A24/MAGE-4 tetramers. Approximately 10⁵ cells from each microculture were labeled for 15 minutes at room temperature with the A24/MAGE-4 tetramer (20 nM) and for 15 minutes with an anti-CD8 antibody. The samples were then analyzed by flow cytometry, according to standard procedures. Three microcultures were found to contain tetramer-positive cells, indicating that T cells in these microcultures expressed a T cell receptor (TCR) specific for the A24/MAGE-4 peptide complexes. A tetramer-positive CD8⁺ T cell clone (CTL 13) was successfully obtained according to standard limiting dilution procedures. See FIG. 2. This clone was tested for lytic activity, as described in the next example.

### EXAMPLE 5

In this experiment, CTL 13 was tested for specific lytic activity in standard chromium release assays.

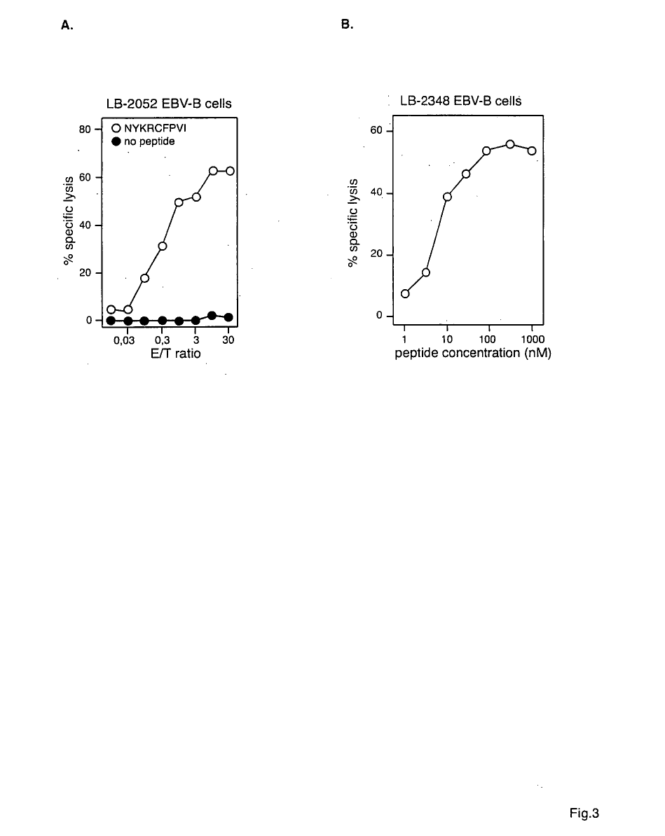

In the assay, the targets of CTL 13 were HLA-A24⁺ Epstein Barr Virus-transformed B (EBV-B) cells. The EBV-B cells were cultured in IMDM supplemented with 10% fetal calf serum, 0.24 mM L-asparagine, 0.55 mM L-arginine, AAG, 100 U/ml penicillin and 100 μg/ml streptomycin. The EBV-B cells were labeled with ⁵¹Cr for one hour according to standard methods and then incubated for 5 minutes with peptide NYKRCFPVI (SEQ ID NO: 1) (1 μg/ml). The ⁵¹Cr labeled, peptide pulsed EBV-B cells (targets) were then combined with CTL 13 (effector) at effector-to-target ratios shown in FIG. 3A. Chromium release was measured after 4 hours. The results showed that CTL 13 had specific anti-MAGE-4 lytic activity. CTL 13 lysed target cells pulsed with SEQ ID NO: 1 peptide but not unpulsed control target cells.

In further experiments, the HLA-A24 EBV-B cells were labeled with ⁵¹Cr for one hour according to standard methods and then incubated for 15 minutes with threefold dilutions of the synthetic peptide NYKRCFPVI (SEQ ID NO: 1). See FIG. 3B. CTL 13 was subsequently added at an effector-to-target ratio of 10:1 and chromium release was measured after 4 hours. The concentration of peptide shown in FIG. 3B corresponds to the concentrations during the 4 hour incubation. The results showed that half maximal lysis of the peptide pulsed HLA-A24 target cells was obtained at a peptide concentration of 8 nM. This is in the range of concentration observed for the previously identified antigenic MAGE peptides, for which values ranging from 0.05 to 200 nM have been observed. See Kobayashi, et al., supra; Chaux, et al., supra; Schultz, et al., Tissue Antigens 57:103-109 (2001); Luiten, et al., supra; Traversari, et al., J. Exp. Med. 176:1453-1457 (1992); Van der Bruggen, et al., Eur. J. Immunol. 24:2134-2140 (1994), all incorporated by reference.

### EXAMPLE 6

The following experiments were conducted to determine recognition of cells expressing the MAGE-4 protein.

COS-7 cells (1.5×10⁴) were distributed in flat-bottom microwells and were maintained in DMEM supplemented with 5% fetal calf serum, 0.24 mM L-asparagine, 0.55 mM L-arginine, AAG, 100 U/ml penicillin and 100 μg/ml streptomycin. The cells were co-transfected one day later with MAGE-4 cDNA inserted into expression vector pcDNAI/Amp and cDNA encoding HLA-A*2402 inserted into expression vector pcDNA3. Transfections were performed with 15,000 COS-7 cells, 50 ng of each cDNA and 1 μl of lipofectamine, in accordance with standard methods. Control COS-7 cells were transfected with either the MAGE-4 construct or the HLA-A24 construct. Cells that were not transfected with either MAGE-4 or HLA-A24 were used as an additional control.

The transfected cells were incubated for 24 hours at 37° C. and 8% CO₂. The transfectants were then tested for their ability to stimulate the production of tumor necrosis factor (TNF) by CTL 13. Three thousand CTL 13 were added to the microwells containing the COS-7 transfectants, in a total volume of 150 μl of complete IMDM supplemented with 25 U/ml of IL-2. After 24 hours, IFN-γ production was measured by ELISA, according to standard procedures using commercially available reagents.

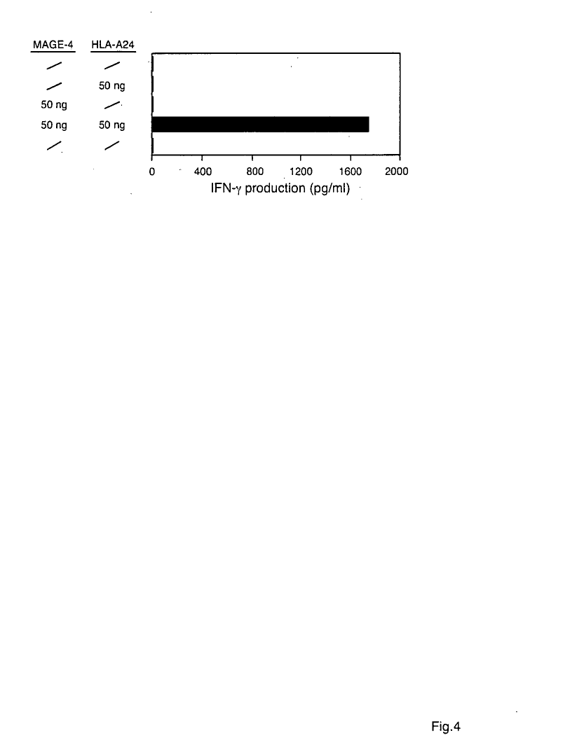

Only COS-7 cells that had been co-transfected with the MAGE-4 and HLA-A2402 cDNA constructs stimulated CTL 13 to produce IFN-γ. See FIG. 4. This indicates that the MAGE-4 antigenic peptide (SEQ ID NO: 1) could be processed and presented in these cells for recognition by the A24 restricted MAGE-4 peptide specific T cell clone, CTL 13. The control cells did not stimulate IFN-γ production, confirming the antigen specificity and HLA restriction of the CTL 13 clone.

### EXAMPLE 7

Since COS-7 cells expressing MAGE-4 had been used to activate CTL 13, the following experiments were designed to further verify that tumor cells could also naturally process and present the MAGE-4.A24 antigen.

Tumor cells from two cell lines were used. The cell lines were derived from a patient who had been typed for HLA-A2402 and expressed MAGE-4. K562 cells were used as a control because these cells are targets for natural killer cells.

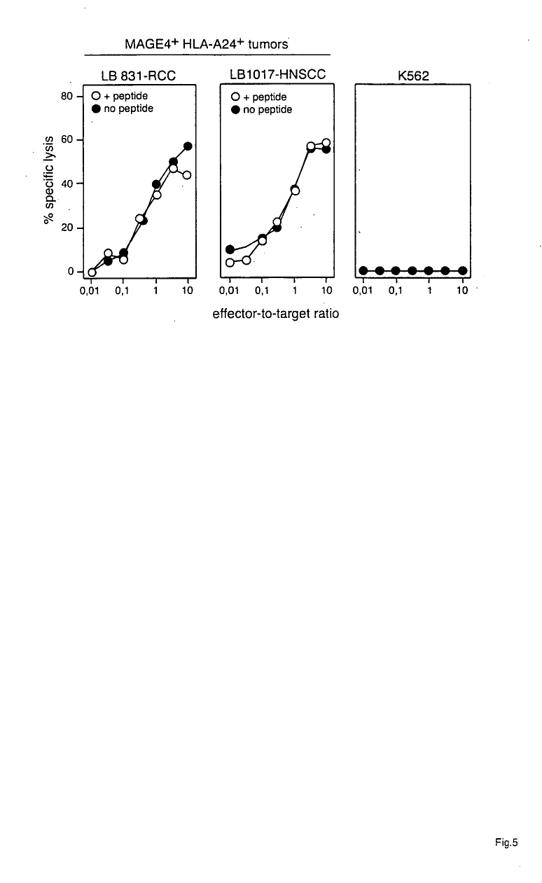

The tumor cells were treated with 50 U/ml IFN-γ for 7 days. The tumor and control cells were then labeled with 100 μCi of Na(51Cr)O for one hour, and as indicated in FIG. 5, pulsed for 5 minutes with peptide NYKRCFPVI (SEQ ID NO: 1) (1 μg/ml). The labeled cells were incubated at 37° C. for 4 hours with CTL 13 at the effector-to-target ratios indicated in FIG. 5 and chromium release was measured. The results showed that both tumor cell lines were lysed by CTL 13, indicating that they naturally processed the MAGE-4.A24 peptide. Control K562 cells were not lysed.

The foregoing disclosure sets forth various features of the invention. These include isolated peptides which are processed to peptides that form immunogenic complexes with HLA-A24 molecules. The peptides of the invention comprise the amino acid sequence set forth in SEQ ID NO: 1:
NYKRCFPVI
   concatenated to from 1 to about 31 additional amino acids at the N (Asn) or I (Ile) terminus, preferably from 5-10 additional amino acids. Preferably, the concatenated amino acids are identical to the amino acid sequence which precedes Asn or follows Ile in the full length amino acid sequence of MAGE-4, but the concatenated amino acids also accommodate variations, such as conservative substitutions, deletions, additions and so forth. The peptides of the invention possess the functional properties of being taken up by antigen presenting cells, such as dendritic cells, and being processed to the 9 amino acid sequence described supra. Preferably, the cells which take up the peptides are cells which present HLA-A24 molecules on their surface.

Also a feature of this invention are isolated T cells, preferably cytolytic T cells, which are specific for complexes of HLA-A24 molecules and amino acid sequences comprising SEQ ID NO: 1, preferably the amino acid sequence of SEQ ID NO: 1, referred to supra, which do not recognize other complexes, including complexes of the sequence and different HLA molecules. As was shown, supra, such cytolytic T cells can be prepared using standard methodologies, including those described herein.

In connection with the cytolytic T cells of the invention, various methods can be used to identify and to secure these. Such methodologies include, i.e., FACS or other analytical methods, preferably in combination with molecules, such as tetrameric compounds of avidin or streptavidin, biotin, and HLA/peptide complexes, to identify relevant T cells from samples.

The ability of the peptides to form recognizable complexes makes them useful as therapeutic agents in conditions such as cancer, including melanoma, where the peptide forms a complex with the HLA molecule, leading to recognition by T cells such as a CTL, and lysis thereby. As was shown, supra, T cells which recognize the complexes occur naturally in patients, so administration of the peptide of the invention to an HLA-A24 positive subject in need of a cytolytic T cell response is another feature of the invention. Such subjects may be, e.g., cancer patients. Such patients may receive the peptide of the invention, or “cocktails” which comprise more than one peptide, as long as the peptide cocktail includes the peptide of the invention. The peptide component of such cocktails may consist of the peptides described herein, or may combine some peptides disclosed herein with other peptides known in the art, such as the following, which bind to Class I or Class II MHC.

| | | | SEQ ID |
|---|---|---|---|
| PEPTIDE SEQUENCE | ANTIGEN | HLA | NO: |
| YMDGTMSQV | TYROSINASE | A2 | 4 |
| MLLAVLYCL | TYROSINASE | A2 | 5 |
| EAAGIGILTV | MELAN-A | A2 | 6 |
| IMPKAGLLI | MAGE-A3 | A24 | 7 |
| FLWGPRALV | MAGE-A3 | A2 | 8 |
| VRIGHLYIL | MAGE-A12 | Cw7 | 9 |
| YLQLVFGIEV | MAGE-A2 | A2 | 10 |
| FLWGPRALV | MAGE-A12 | A2 | 11 |
| VLPDVFIRC(V) | GnTV | A2 | 12 |
| KASEKIFYV | SSX2 | A2 | 13 |
| GLYDGMEHL | MAGE-A10 | A2 | 14 |
| EVDPIGHLY | MAGE-A3 | A1 | 15 |
| SLLMWITQC | NY-ESO-1 | A2 | 16 |
| IMPKAGLLI | MAGE-A3 | A24 | 17 |
| EVDPIGHLY | MAGE-A3 | B35 | 18 |
| GVYDGREHTV | MAGE-A4 | A2 | 19 |
| EADPTGHSY | MAGE-A1 | A1, B35 | 20 |
| SEIWRDIDF | TYROSINASE | B44 | 21 |
| LPSSADVEF | TYROSINASE | B35 | 22 |
| MEVDPIGHLY | MAGE-A3 | B18, B44 | 23 |
| YRPRPRRY | GAGE-1, 2, 8 | Cw6 | 24 |
| LAMPFATPM | NY-ESO-1 | Cw3 | 25 |
| ARGPESRLL | NY-ESO-1 | Cw6 | 26 |
| YYWPRPRRY | GAGE-3, 4, 5, 6, 7 | A29 | 27 |
| AARAVFLAL | BAGE-1 | Cw16 | 28 |
| TQHFVQENYLEY | MAGE-A3 | DP4 | 29 |
| SLLMWITQCFL | NY-ESO-1 | DP4 | 30 |
| AELVHFLLLKYRAR | MAGE-A3 | DR13 | 31 |
| LLKYRAREPVTKAE | MAGE-A3, A6, A2 | DR13 | 32 |
| AELVHFLLLKYRAR | MAGE-A-12 | DR13 | 33 |
| EYVIKVSARVRF | MAGE-A1 | DR15 | 34 |
| LLKYRAREPVTKAE | MAGE-A1 | DR13 | 35 |
| PGVLLKEFTVSGNILTIRLT | NY-ESO-1 | DR4 | 36 |
| AADHRQLQLSISSCLQQL | NY-ESO-1 | DR4 | 37 |

In an especially preferred embodiment, one administers a cocktail of peptides based upon the HLA profile of the subject being treated. Based upon known Class I peptide binding motifs, such as those set forth by Rammensee, et al., supra, peptides such as those set forth at SEQ ID NOS: 4-37 would be expected to bind to other HLA-Class I or II alleles, such as HLA-A1, A3, B7, B8, B15, B27, B44, B51 in addition to HLA-A2, and subtypes thereof. Further, if appropriate, one or more peptides which bind to HLA-A2, HLA-B7, HLA-A24 and so forth, can be admixed, preferably in the presence of an adjuvant like GM-CSF, alum, Montanide or other adjuvants well known to the art, such as CpG. See U.S. Pat. Nos. 6,339,068; 6,239,116; 6,207,646 and 6,194,388, all of which are incorporated by reference. Also possible as therapeutic agents are peptide pulsed, autologous dendritic cells. See, e.g., Jonuleit, et al., Int. J. Cancer 93(2):243-51 (2001); Schuler-Thumer, et al., J. Immunol 165(6):3492-6 (2000); Thumer, et al., J. Exp. Med. 190(11):1669-78 (1999), all of which are incorporated by reference and show, e.g., the use of peptide pulsed dendritic cells as vaccines and as adjuvants. Such combinations of peptides, in the form of compositions, are another feature of the invention, either alone or in combination with such adjuvants. Similarly, one can administer T cells specific for the peptide/HLA-A24 complexes, such as autologous CTLs, which can be prepared as described in the preceding examples or other methods well known in the art. These CTLs, which are specific for complexes of the 9 amino acid molecules described supra and HLA-A24, and no other complexes, are a further feature of the invention. Administration of soluble T cell receptors derived from such CTLs linked to toxins and/or cytotoxic drugs and radiolabels is also contemplated

Yet a further feature of the invention are nucleic acid molecules which consist of nucleotide sequences that encode the peptides of the invention. Such nucleic acid molecules may be used to encode the peptides of the invention, and may be combined into expression vectors, operably linked to a promoter. More than one sequence can be combined in such expression vectors, as can nucleic acid molecules which encode HLA-A24 molecules. The constructs can be used to transfect cells, so as to generate the CTLs, or for administration to subjects in need of a cytolytic T cell response or augmentation of a pre-existing T cell response. Such administration could be one of, e.g., administering vector constructs as described, heterologous expression vectors, peptides or recombinant proteins, such as the full length proteins, preferably in recombinant form, from which one or more of the peptides are derived as discussed supra. Expression vectors include recombinant viral vectors such as Vaccinia virus, pox virus, adenovirus, lentiviruses, retroviral and other viral vectors known in the art.

The invention also relates to the use of the peptides, CTLs, and other, immunologically active components, such as antibodies, as well as T cell receptors, such as soluble T cell receptors, to diagnose pathological conditions such as cancer, e.g, melanoma, or cancers expressing MAGE-4 in particular. As was shown, supra, MAGE-4 is expressed in cancer cells and the presence of complexes of the 9 mer and HLA-A24 is indicative of a pathological condition. By determining the interaction of the immunologically active component and the complex (by way of, e.g., antibody binding, soluble T cell receptor binding, TNF release, cell lysis, etc.), one can diagnose the pathology, or even determine the status of the pathology via comparing a value to a pre-existing value for the same parameter.

Also a part of this invention are antibodies, e.g., polyclonal and monclonal, and antibody fragments, e.g., single chain Fv, Fab, diabodies, etc., that specifically bind the peptides or HLA/peptide complexes disclosed herein. Preferably, the antibodies, the antibody fragments and T cell receptors bind the HLA/peptide complexes in a peptide-specific manner. Such antibodies are useful, for example, in identifying cells presenting the HLA/peptide complexes. Such antibodies are also useful in promoting the regression or inhibiting the progression of a tumor which expresses complexes of the HLA and peptide. Polyclonal antisera and monoclonal antibodies specific to the peptides or HLA/peptide complexes of this invention may be generated according to standard procedures. See e.g., Catty D., Antibodies, A Practical Approach, Vol. 1, IRL Press, Washington D.C. (1988); Klein J., Immunology: The Science of Cell-Non-Cell Discrimination, John Wiley and Sons, New York (1982); Kennett, R., et al., Monoclonal Antibodies, Hybridoma, A New Dimension In Biological Analyses, Plenum Press, New York (1980); Campbell, A., Monoclonal Antibody Technology, in Laboratory Techniques and Biochemistry and Molecular Biology, Vol. 13 (Burdon, R. et al. EDS.), Elsevier Amsterdam (1984); Eisen, H. N., Microbiology, third edition, Davis, B. D. et al. EDS. (Harper & Rowe, Philadelphia (1980); Kohler and Milstein, Nature, 256:495 (1975) all incorporated herein by reference.) Methods for identifying Fab molecules endowed with the antigen-specific, HLA-restricted specificity of T cells has been described by Denkberg et al., PNAS 99:9421-9426 (2002) and Cohen et al., Cancer Research 62:5835-5844 (2002), both incorporated herein by reference. Methods for generating and identifying other antibody molecules, e.g., scFv, phage libraries diabodies are well known in the art, see e.g., Bird et al., Science, 242:423-426 (1988); Huston et al., Proc. Natl. Acad. Sci., 85:5879-5883 (1988); Mallender and Voss, J. Biol. Chem. 269:199-206 (1994); Ito and Kurosawa, J. Biol Chem. 27: 20668-20675 (1993), and Gandecha et al., Prot Express Purif. 5: 385-390 (1994).

The antibodies of this invention can be used for experimental purposes (e.g. localization of the HLA/peptide complexes, immunoprecipitations, Western Blots, flow cytometry, ELISA etc.) as well as diagnostic or therapeutic purposes, e.g., assaying extracts of tissue biopsies for the presence of HLA/peptide complexes, targeting delivery of cytotoxic or cytostatic substances to cells expressing the appropriate HLA/peptide complex. The antibodies of this invention are useful for the study and analysis of antigen presentation on tumor cells and can be used to assay for changes in the HLA/peptide complex expression before, during or after a treatment protocol, e.g., vaccination with peptides, antigen presenting cells, HLA/peptide tetramers, adoptive transfer or chemotherapy. The antibodies and antibody fragments of this invention may be coupled to diagnostic labeling agents for imaging of cells and tissues that express the HLA/peptide complexes or may be coupled to therapeutically useful agents by using standard methods well-known in the art. The antibodies also may be coupled to labeling agents for imaging e.g., radiolabels or fluorescent labels, or may be coupled to, e.g., biotin or antitumor agents, e.g., radioiodinated compounds, toxins such as ricin, methotrexate, cytostatic or cytolytic drugs, etc. Examples of diagnostic agents suitable for conjugating to the antibodies of this invention include e.g., barium sulfate, diatrizoate sodium, diatrizoate meglumine, iocetamic acid, iopanoic acid, ipodate calcium, metrizamide, tyropanoate sodium and radiodiagnostics including positron emitters such as fluorine-18 and carbon-11, gamma emitters such as iodine-125, technitium-99m, iodine-131 and indium-111, nuclides for nuclear magnetic resonance such as fluorine and gadolinium. As used herein, “therapeutically useful agents” include any therapeutic molecule which are preferably targeted selectively to a cell expressing the HLA/peptide complexes, including antineoplastic agents, radioiodinated compounds, toxins, other cytostatic or cytolytic drugs. Antineoplastic therapeutics are well known and include: aminoglutethimide, azathioprine, bleomycin sulfate, busulfan, carmustine, chlorambucil, cisplatin, cyclophosphamide, cyclosporin, cytarabidine, dacarbazine, dactinomycin, daunorubicin, doxorubicin, taxol, etoposide, fluorouracil, interferon-.alpha., lomustine, mercaptopurine, methotrexate, mitotane, procarbazine HCl, thioguanine, vinblastine sulfate and vincristine sulfate. Additional antineoplastic agents include those disclosed in Chapter 52, Antineoplastic Agents (Paul Calabresi and Bruce A. Chabner), and the introduction thereto, 1202-1263, of Goodman and Gilman's “The Pharmacological Basis of Therapeutics”, Eighth Edition, 1990, McGraw-Hill, Inc. (Health Professions Division). Toxins can be proteins such as, for example, pokeweed anti-viral protein, cholera toxin, pertussis toxin, ricin, gelonin, abrin, diphtheria exotoxin, or Pseudomonas exotoxin. Toxin moieties can also be high energy-emitting radionuclides such as cobalt-60. The antibodies may be administered to a subject having a pathological condition characterized by the presentation of the HLA/peptide complexes of this invention, e.g., melanoma and several other cancers, as described in Jungbluth et al., Int. J. Cancer, 92:856-860 (Jun. 15, 2001) (incorporated herein by reference), in an amount sufficient to alleviate the symptoms associated with the pathological condition.

Soluble T cell receptors (sTCRs) which specifically bind to the HLA/peptide complexes described herein are also an aspect of this invention. In their soluble form, T cell receptors are analogous to a monoclonal antibody in that they bind to HLA/peptide complex in a peptide-specific manner. Immobilized TCRs or antibodies may be used to identify and purify unknown peptide/HLA complexes which may be involved in cellular abnormalities. Methods for identifying and isolating sTCRs are known in the art, see for example WO 99/60119, WO 99/60120 (both incorporated herein by reference) which describe synthetic multivalent T cell receptor complex for binding to peptide-MHC complexes. Recombinant, refolded soluble T cell receptors are specifically described. Such receptors may be used for delivering therapeutic agents or detecting specific peptide-MHC complexes expressed by tumor cells. WO 02/088740 (incorporated by reference) describes a method for identifying a substance that binds to a peptide-MHC complex. A peptide-MHC complex is formed between a predetermined MHC and peptide known to bind to such predetermined MHC. The complex is then used to screen or select an entity that binds to the peptide-MHC complex such as a T cell receptor. The method could also be applied to the selection of monoclonal antibodies that bind to the predetermined peptide-MHC complex.

Also an embodiment of this invention are nucleic acid molecules encoding the antibodies and T cell receptors of this invention and host cells, e.g., human T cells, transformed with a nucleic acid molecule encoding a recombinant antibody or antibody fragment, e.g., scFv or Fab, or a TCR specific for a pre-designated HLA/peptide complex as described herein. Recombinant Fab or TCR specific for a pre-designated HLA/peptide complex in T cells have been described in, e.g., Willemsen et al., “A phage display selected fab fragment with MHC class I-restricted specificity for MAGE-A1 allows for retargeting of primary human T lymphocytes” Gene Ther. 2001 November;8(21):1601-8. PMID: 11894998 and Willemsen et al., “Grafting primary human T lymphocytes with cancer-specific chimeric single chain and two chain TCR”. Gene Ther. 2000 August;7(16):1369-77. PMID: 10981663 (both incorporated herein by reference) and have applications in an autologous T cell transfer setting. The autologous T cells, transduced to express recombinant antibody or a T cell receptor or T cell receptor chain, such as an sTCR, a TCRα chain or TCRβ chain, may be infused into a patient having an pathological condition associated with cells expressing the HLA/peptide complex. The transduced T cells are administered in an amount sufficient to inhibit the progression or alleviate at least some of the symptoms associated with the pathological condition.

An embodiment of this invention is a method for promoting regression or inhibiting progression of a tumor in a subject in need thereof wherein the tumor expresses a complex of HLA and peptide. The method comprises administering an antibody, antibody fragment or soluble T cell receptor, which specifically binds to the HLA/peptide complex, or by administering cells transduced so that they express those antibodies or TCR in amounts that are sufficient to promote the regression or inhibit progression of the tumor expressing the HLA/peptide complex, e.g., a melanoma or other cancer. The antibodies, antibody fragments and soluble T cell receptors may be conjugated with, or administered in conjunction with, an antineoplastic agent, e.g., radioiodinated compounds, toxins such as ricin, methotrexate, or a cytostatic or cytolytic agent as discussed supra. See e.g., Patan et al., Biochem. Biophys. Acta, 133:C₁-C₆ (1997), Lode et al., Immunol. Res. 21:279-288 (2000) and Wihoff et al., Curr. Opin. Mo. Ther. 3:53-62 (2001) (all incorporated herein by reference) for a discussion of the construction of recombinant immunotoxins, antibody fusions with cytokine molecules and bispecific antibody therapy or immunogene therapy.

The invention also embraces functional variants of the MAGE-4 HLA class I binding peptide. As used herein, a “functional variant” or “variant” of a MAGE-4 HLA class I binding peptide is a peptide which contains one or more modifications to the primary amino acid sequence of MAGE-4 HLA class I binding peptide and retains the HLA class I and T cell receptor binding properties disclosed herein. Modifications which create a MAGE-4 HLA class I binding peptide functional variant can be made for example: 1) to enhance a property of a MAGE-4 HLA class I binding peptide, such as peptide stability in an expression system or the stability of protein-protein binding such as HLA-peptide binding; 2) to provide a novel activity or property to a MAGE-4 HLA class I binding peptide, such as addition of an antigenic epitope or addition of a detectable moiety; or 3) to provide a different amino acid sequence that produces the same or similar T cell stimulatory properties. Modifications to a MAGE-4 HLA class I binding peptide can be made to a nucleic acid which encodes the peptide, and can include deletions, point mutations, truncations, amino acid substitutions and additions of amino acids. Alternatively, modifications can be made directly to the polypeptide, such as by cleavage, addition of a linker molecule, addition of a detectable moiety, such as biotin, addition of a fatty acid, substitution of one amino acid for another and the like. Modifications also embrace fusion proteins comprising all of part of the MAGE-4 HLA class I binding peptide amino acid sequence.

The amino acid sequence of MAGE-4 HLA class I binding peptides may be of natural or non-natural origin, that is, they may comprise a natural MAGE-4 HLA class I binding peptide molecule or may comprise a modified sequence as long as the amino acid sequence retains the ability to stimulate T cells when presented and retains the property of binding to an HLA class I molecule such as an HLA-A24 molecule. For example, MAGE-4 HLA class I binding peptides in this context may be fusion proteins of a MAGE-4 HLA class I binding peptide and unrelated amino acid sequences, a synthetic peptide of the amino acid sequence shown in SEQ ID NO: 1, labeled peptides, peptides isolated from patients with a MAGE-4 expressing cancer, peptides isolated from cultured cells which express MAGE-4, peptides coupled to nonpeptide molecules (for example in certain drug delivery systems) and other molecules which include the amino acid sequence of SEQ ID NO: 1.

Preferably, MAGE-4 HLA class I binding peptides are non-hydrolyzable. To provide such peptides, one may select MAGE-4 HLA class I binding peptides from a library of non-hydrolyzable peptides, such as peptides containing one or more D-amino acids or peptides containing one or more non-hydrolyzable peptide bonds linking amino acids. Many non-hydrolyzable peptide bonds are known in the art, along with procedures for synthesis of peptides containing such bonds. Non-hydrolyzable bonds include—psi[Ch.sub.2 NH]-reduced amide peptide bonds, -psi[COCH.sub.2]-ketomethylene peptide bonds, -psi[CH(CN)NH]-(cyanomethlylene) amino peptide bonds, -psi[CH.sub.2CH(OH)]-hydroxyethylene peptide bonds, -psi[CH.sub.2 O]-peptide bonds, and -psi[CH.sub.2 S]-thiomethylene peptide bonds. Methods for determining such functional variants are provided in U.S. Pat. No. 6,087,441, incorporated by reference.

If a variant involves a change to the amino acid of SEQ ID NO: 1, functional variants of the MAGE-4 HLA class I binding peptide having conservative amino acid substitutions typically will be preferred, i.e., substitutions which retain a property of the original amino acid such as charge, hydrophobicity, conformation, etc. Examples of conservative substitutions of amino acids include substitutions made amongst amino acids within the following groups: (a) M, I, L, V; (b) F, Y, W: (c) K, R, H; (d) A, G; (e) S, T: (f) Q, N; and (g) E, D. Methods for identifying functional variants of the MAGE-4 HLA class I binding peptides are provided in a U.S. Pat. Nos. 6,277,956 and 6,326,200 and published PCT application WO0136453 (U.S. patent application Ser. Nos. 09/440,621, 09/514,036, 09/676,005), all of which are incorporated by reference.

Thus, methods for identifying functional variants of a MAGE-4 HLA class I binding peptide are provided. In general, the methods include selecting a MAGE-4 HLA class I binding peptide, an HLA class I binding molecule which binds the MAGE-4 HLA class I binding peptide, and a T cell which is stimulated by the MAGE-4 HLA class I binding peptide presented by the HLA class I binding molecule. In preferred embodiments, the MAGE-4 HLA class I binding peptide comprises the amino acid sequence set forth in SEQ ID NO: 1. A first amino acid residue of the MAGE-4 HLA class I binding peptide is mutated to prepare a variant peptide. Any method for preparing variant peptides can be employed, such as synthesis of the variant peptide, recombinantly producing the variant peptide using a mutated nucleic acid molecule, and the like.

The binding of the variant peptide to HLA class I binding molecule and stimulation of the T cell are then determined according to standard procedures wherein binding of the variant peptide to the HLA class I binding molecule and stimulation of the T cell by the variant peptide presented by the HLA class I binding molecule indicates that the variant peptide is a functional variant. For example, the variant peptide can be contacted with an antigen presenting cell which contains the HLA class I molecule which binds the MAGE-4 peptide to form a complex of the variant peptide and antigen presenting cell. This complex can then be contacted with a T cell which recognizes the epitope formed by the MAGE-4 HLA class I binding peptide and the HLA class I binding molecule. T cells can be obtained from a patient having a condition characterized by expression of MAGE-4. Recognition of variant peptides by the T cells can be determined by measuring an indicator of T cell stimulation.

Binding of the variant peptide to the HLA class I binding molecule and stimulation of the T cell by the epitope presented by the complex of variant peptide and HLA class I binding molecule indicates that the variant peptide is a functional variant. The methods also can include the step of comparing the stimulation of the T cell by the epitope formed by the MAGE-4 HLA class I binding peptide and the HLA class I molecule, stimulation of the T cell as a determination of the effectiveness of the stimulation of the T cell by the epitope. By comparing the epitope involving the epitope formed by the functional variant with the MAGE-4 HLA class I binding peptide, peptides with increased T cell stimulatory properties can be prepared.

Variants of the MAGE-4 HLA class I binding peptides prepared by any of the foregoing methods can be sequenced, if necessary, to determine the amino acid sequence and thus deduce the nucleotide sequence which encodes such variants.

Other features of the invention will be clear to the skilled artisan, and need not be reiterated herein.

1. An isolated peptide, consisting of from 10 to about 40 amino acids, wherein the amino acid sequence of said peptide consists of
NYKRCFPVI
concatenated to from 1 to about 31 additional amino acids at N or I, wherein said peptide is processed by an antigen presenting cell to a peptide which binds to an MHC molecule and stimulates production of cytolytic T cells which recognize the presented tumor rejection antigen.

2. The isolated peptide of claim 1, wherein the amino acid sequence of said peptide is found in the amino acid sequence of MAGE-4.

3. The isolated peptide of claim 1, wherein said MHC molecule is an HLA molecule.

4. The isolated peptide of claim 3, wherein said HLA molecule is an HLA-Class I molecule.

5. The isolated peptide of claim 4, wherein said HLA-Class I molecule is an HLA-A24 molecule.

6. The isolated peptide of claim 1, wherein said peptide is processed to a peptide, the amino acid sequence of which is set forth in SEQ ID NO: 1.

7. An isolated nucleic acid molecule consisting of a nucleotide sequence which encodes the isolated peptide of claim 1.

8. An isolated nucleic acid molecule consisting of a nucleotide sequence which encodes a peptide consisting of the amino acid sequence set forth in SEQ ID NO: 1.

9. A composition useful in stimulating a cytolytic T cell response, comprising the isolated peptide of claim 1 and an adjuvant.

10. A method for treating a subject with a pathological condition whose cells characteristic of said pathological condition present HLA-A24 molecules on their surface, comprising administering to said subject an amount of a peptide of claim 1, wherein said amount is sufficient to generate a therapeutically effective, immunologically active response against said cells.

11. A method for treating a subject with a pathological condition whose cells characteristic of said pathological condition present HLA-A24 molecules on their surface, comprising administering to said subject an amount of a peptide, which consists of the amino acid sequence set forth in SEQ ID NO: 1, wherein said amount is sufficient to generate a therapeutically effective, immunologically active response against said cells.

12. The method of claim 10, comprising administering said peptide in combination with at least one additional peptide which forms a complex with an MHC molecule other than HLA-A24.

13. The method of claim 10, comprising administering said peptide in combination with an adjuvant.

14. The method of claim 10, wherein said immunologically active response is a cytolytic T cell response which causes lysis of cells that present complexes of said peptide and HLA-A24 molecules on their surfaces.

15. The method of claim 10, wherein said pathological condition is cancer.

16. The method of claim 15, wherein said cancer is esophagus cancer, head and neck cancer, lung cancer, bladder cancer or breast cancer.

17. A method for treating a subject with a pathological condition, whose cells characteristic of said pathological condition present complexes of HLA-A24 molecules and a peptide consisting of the amino acid sequence set forth in SEQ ID NO: 1 on their surface, comprising administering to said subject an amount of cytolytic T lymphocytes specific for said complexes, sufficient to lyse cells presenting said complexes.

18. The method of claim 17, wherein said cytolytic T lymphocytes are autologous T lymphocytes.

19. The method of claim 17, wherein said pathological condition is cancer.

20. The method of claim 19, wherein said cancer is esophagus cancer, head and neck cancer, lung cancer, bladder cancer or breast cancer.

21. An isolated cytolytic T lymphocyte which recognizes complexes of HLA-A24 molecules and the peptide consisting of the amino acid sequence set forth in SEQ ID NO: 1, wherein said isolated cytolytic T lymphocyte does not recognize any other complexes of MHC molecules and peptides.

22. A method for determining if a subject is suffering from a pathological condition, comprising administering a sample of cells taken from said subject with an immunologically active agent which recognizes complexes of an HLA-A24 molecule and the peptide consisting of the amino acid sequence set forth in SEQ ID NO: 1, and determining interaction between said immunologically active agent and said complexes, wherein interaction indicates the subject suffers from said pathological condition.

23. The method of claim 22, wherein said immunologically active agent is a cytolytic T lymphocyte.

24. The method of claim 23, comprising determining lysis of cells by said cytolytic T lymphocyte.

25. The method of claim 23, comprising measuring tumor necrosis factor release by said cytolytic T lymphocyte.

26. The method of claim 22, wherein said immunologically active agent is an antibody.


SEQUENCE LISTING (37 sequences)

SEQ ID NO: 1 - protein, 9 aa, Artificial Sequence
  MAGE-4 peptide
NYKRCFPVI

SEQ ID NO: 2 - protein, 9 aa, Artificial Sequence
  PRAME peptide
LYVDSLFFL

SEQ ID NO: 3 - protein, 9 aa, Artificial sequence
  MAGE-1 peptide
NYKHCFPEI

SEQ ID NO: 4 - protein, 9 aa, Artificial sequence
  tyrosinase peptide
YMDGTMSQV

SEQ ID NO: 5 - protein, 9 aa, Artificial Sequence
  tyrosinase peptide
MLLAVLYCL

SEQ ID NO: 6 - protein, 10 aa, Artificial sequence
  Melan-A peptide
EAAGIGILTV

SEQ ID NO: 7 - protein, 9 aa, Artificial sequence
  MAGE-A3 peptide
IMPKAGLLI

SEQ ID NO: 8 - protein, 9 aa, Artificial sequence
  MAGE-A3 peptide
FLWGPRALV

SEQ ID NO: 9 - protein, 9 aa, Artificial sequence
  MAGE-A12 peptide
VRIGHLYIL

SEQ ID NO: 10 - protein, 10 aa, Artificial sequence
  MAGE-A2 peptide
YLQLVFGIEV

SEQ ID NO: 11 - protein, 9 aa, artificial sequence
  MAGE-A12 peptide
FLWGPRALV

SEQ ID NO: 12 - protein, 10 aa, artificial sequence
  GnTV peptide
VLPDVFIRCV

SEQ ID NO: 13 - protein, 9 aa, artificial sequence
  SSX2 peptide
KASEKIFYV

SEQ ID NO: 14 - protein, 9 aa, artificial sequence
  MAGE-A10 peptide
GLYDGMEHL

SEQ ID NO: 15 - protein, 9 aa, artificial sequence
  MAGE-A3 peptide
EVDPIGHLY

SEQ ID NO: 16 - protein, 9 aa, artificial sequence
  NY-ESO-1 peptide
SLLMWITQC

SEQ ID NO: 17 - protein, 9 aa, artificial sequence
  MAGE-A3 peptide
IMPKAGLLI

SEQ ID NO: 18 - protein, 9 aa, artificial sequence
  MAGE-A3 peptide
EVDPIGHLY

SEQ ID NO: 19 - protein, 10 aa, artificial sequence
  MAGE-A4 peptide
GVYDGREHTV

SEQ ID NO: 20 - protein, 9 aa, artificial sequence
  MAGE-A1 peptide
EADPTGHSY

SEQ ID NO: 21 - protein, 9 aa, artificial sequence
  tyrosinase peptide
SEIWRDIDF

SEQ ID NO: 22 - protein, 9 aa, artificial sequence
  tyrosinase peptide
LPSSADVEF

SEQ ID NO: 23 - protein, 10 aa, artificial sequence
  MAGE-A3 peptide
MEVDPIGHLY

SEQ ID NO: 24 - protein, 8 aa, artificial sequence
  GAGE-1, 2, 8 peptide
YRPRPRRY

SEQ ID NO: 25 - protein, 9 aa, artificial sequence
  NY-ESO-1 peptide
LAMPFATPM

SEQ ID NO: 26 - protein, 9 aa, artificial sequence
  NY-ESO-1 peptide
ARGPESRLL

SEQ ID NO: 27 - protein, 9 aa, artificial sequence
  GAGE-3, 4, 5, 6, 7 peptide
YYWPRPRRY

SEQ ID NO: 28 - protein, 9 aa, artificial sequence
  BAGE-1 peptide
AARAVFLAL

SEQ ID NO: 29 - protein, 12 aa, artificial sequence
  MAGE-A3 peptide
TQHFVQENYLEY

SEQ ID NO: 30 - protein, 11 aa, artificial sequence
  NY-ESO-1 peptide
SLLMWITQCFL

SEQ ID NO: 31 - protein, 14 aa, artificial sequence
  MAGE-A3 peptide
AELVHFLLLKYRAR

SEQ ID NO: 32 - protein, 14 aa, artificial sequence
  MAGE-A2, A6, A2 peptide
LLKYRAREPVTKAE

SEQ ID NO: 33 - protein, 14 aa, artificial sequence
  MAGE-A-12 peptide
AELVHFLLLKYRAR

SEQ ID NO: 34 - protein, 12 aa, artificial sequence
  MAGE-A1 peptide
EYVIKVSARVRF

SEQ ID NO: 35 - protein, 14 aa, artificial sequence
  MAGE-A1 peptide
LLKYRAREPVTKAE

SEQ ID NO: 36 - protein, 20 aa, artificial sequence
  NY-ESO-1 peptide
PGVLLKEFTVSGNILTIRLT

SEQ ID NO: 37 - protein, 18 aa, artificial sequence
  NY-ESO-1 peptide
AADHRQLQLSISSCLQQL